(19) United States
(12) Patent Application Publication
(10) Pub. No.: US 2010/0112040 A1
(43) Pub. Date: May 6, 2010

(54) Preparation of Heavy Metal-Containing Nano-Liposomes and their Uses in Medical Therapy

(75) Inventor: Sobhi Basheer, Sakhnin (IL)

Correspondence Address: MDIP LLC, POST OFFICE BOX 2630, MONTGOMERY VILLAGE, MD, 20886-2630, US

(73) Assignee: S.B. BIOTECHNOLOGIES LTD., Shfar-Am (IL)

(21) Appl. No.: 12/513,202

(22) PCT Filed: Oct. 31, 2007
(86) PCT No.: PCT/IL07/01330

§ 371 Date: Dec. 16, 2009

(30) Foreign Application Priority Data
Nov. 1, 2006 (IL) 178982
Feb. 1, 2007 (IL) 181126

(51) Int. Cl. A61K 9/127; C07F 9/09; A61P 35/00

(52) U.S. Cl. 424/450; 556/13

(57) ABSTRACT

Heavy metal-containing nano-liposome particles and their use in treating, for example, immune-related disorders, such as, cancer and inflammatory conditions, and metal deficiency-related diseases are described. The particles also can be used in diagnostic methods. The particles can contain gold, platinum or iron.

### FIELD OF THE INVENTION

The present invention relates to the field of controlled and targeted release of drugs used in medical therapy, based on nano-liposome technology. More specifically, the present invention relates to a new method of preparing nano-liposome particles in combination with heavy metals, and their use in the treatment of immune-related disorders such as cancer and inflammatory conditions.

### BACKGROUND OF THE INVENTION

All publications mentioned throughout this application are fully incorporated herein by reference, including all references cited therein.

Liposomes are defined as spherical micro- or nano-sized particles that contain an internal water pool surrounded by a bilaminar membrane [Schafer K. M., et al. (1989) J. Am. Acad. Dermatol. 21(6):1271-1275); Van Ballen, et al. (2004) Med. Res. Rev. 24(3):299-324]. The building blocks of the membrane are phospholipid molecules or a combination of phospholipids with sterols, such as cholesterol, or with other surface-active ingredients. Phospholipids are a group of molecules that have both hydrophilic and hydrophobic nature. This grants phospholipid molecules a unique property that is related to the production of complex structures such as membranes or liposomes that may be found throughout every living body. These structures are formed when phospholipids are in an aqueous phase so that the hydrophilic part of these molecules (the head group) faces outward towards the water layer and the hydrophobic part (the tail group) faces the inner side of the organized structure that forms. Under these conditions, the spherical structures known as liposomes usually form with a diameter of 20 nm to 500 microns and with a membrane thickness of 4-7 nm. Nano-liposomes refer exclusively to nanoscale lipid vesicles.

The hydrophilic head group of the phospholipids is what determines the surface properties of the membrane of the liposomes, because it is in direct contact with both the external water and the internal water inside the liposome lumen. Natural standard head groups are mainly choline, ethanolamine, glycerol, serine and inositol. Phosphatidyl choline (PC) is the most common phospholipid for making liposomes.

The source of PC is usually egg lecithin, but there are also vegetable sources such as soybeans. The use of liposomes in controlled drug release has been known to the medical market for more than 5 years. In addition, dozens of different drugs at phase I to phase III clinical trials are based on various liposome preparation technologies [Barros, M. et al. (2001) Biochem. Biophys. Res. Comm. 288:225-232; Galvain, S. et al. (1999) Current, Therapeutic Res. 60(5):277-294; Goyal, P. et al. (2005) Acta Pharm. 55:1-25; Koning, G. A. et al. (1999) Biochem. Biophys. Acta. 1420:153-167]. These technologies usually differ in the mode of binding of the drug to the liposomes. Most of the technologies in this field use natural phospholipids, such as phosphatidyl choline combined with various substances known to stabilize liposomes, such as cholesterol. In most cases, the drug is in a soluble form within the internal water pool of the spherical particle, whereas hydrophobic drugs are dissolved in the lipophilic bilaminar layer of the liposomes. Drug-bearing liposomes are typically intravenously injected into the body and are capable of reaching and penetrating the affected cells [Pekiner, B. et al. (1995) Nutr. Res. 15(6):795-802; Schnyder, A. and Huwyler, J. et al. (2005) J. Amr. Soc. Exp. NeuroTherapy, 2(1):99-107; Sinha, J. et al. (2001) Biomed. Pharmacother, 55:264-271].

The results of many studies indicate inconsistency and unsuitability when using liposomes as transporters for the targeted release of drugs to specific lesion sites in the body [Schnyder (2005) id ibid.; Van Ballen (2004) id ibid.]. In addition, these studies attest to the insertion of drug concentrations into liposomes below the desired concentration, and indicate that these drugs do not reach a suitable dosage for expressing their desired therapeutic action inside the body [Koning (1999) id ibid.]. Other disadvantages of using liposomes that have been made using the state-of-the-art methods include:
1. Heterogeneity in drug concentration (or that of another active principal, e.g. gold nano-particles as in the present invention) in the liposome population;
2. Great difficulty in producing liposomes on an industrial scale;
3. Great dispersal in sizes of liposomes;
4. Concentrations below the pharmacological concentration required for the drug (or, as relevant to the present invention of metal, e.g. gold nano-particles) inside the liposomes that reach the affected site;
5. Unsuitability of a certain liposome system produced for release of a certain drug to be used for releasing another drug;
6. Instability of liposomes transporting drugs in the body;
7. The high price of phospholipids, the building blocks of liposomes, reaching approximately a few thousands of US$/kg. The high cost of phospholipids restricts the use of liposomes to expensive drugs, such as cancer drugs.

In order to overcome some of these obstacles, many research and development works have been performed. Most of those studies have mainly focused on methods for stabilizing liposomes [Oussoren, C., et al. (1998) Biochem. Biophys. Acta (BBA)-Biomembranes. 1370(2):259-272; Samuni, A. et al. (2000) Chem. Physc. Of Lipids. 105:121-134], particularly:
Prevention of decomposition of the liposomes by hydrolysis of the ester bonds in the molecules constituting the membrane.
Prevention of oxidation of the tail groups, which are composed mainly of polyunsaturated carbon chains.
Prevention of decomposition of the liposomes due to light or heat.
Increasing the tolerance for these foreign immunogenic objects when injected into the circulatory system in the living body.

### Nano-Liposomes Containing Heavy Metal Atoms

Because of the high electronic density of gold and the homogeneity in the size and shape of gold atoms, this substance has become attractive for medical applications.

Two technologies have been described for capturing gold atoms in liposomes: [Hong, K. et al. (1983) Biochem. Biophys. Acta. 732:320-323; Huang, S. K. et al. (1991) Biochem. Biophys. Acta (BBA)-Biomembranes. 1069(1):117-1211.
Making liposomes in an aqueous system that contains gold in the form of a water-soluble complex that is dissolved in the presence of a mildly metal reducing substance for turning gold ions into nano-particles of metallic gold atoms.
Making liposomes in an aqueous system containing nano-particles of metallic gold emulsified in the system.

There are many obstacles to making stabilized liposomes that contain a critical number of heavy metal atoms. This limits the use of such atoms, particularly for medical applications. The present invention brings a solution to these obstacles, as described in the following Description and Examples.

Liposomes might be used particularly in brachytherapy, as means to provide targeted release of drugs or metal atoms to the tissue to be treated. Brachytherapy refers to the technique of implanting radioactive sources (seeds) directly into a specific part of the body where a solid tumor to be treated is located. The sources give a high radiation dose to the tumor while reducing the radiation exposure in the surrounding healthy tissues. There are a number of radioactive isotopes that are commonly used as a source of radiation, mainly Iodine¹²⁵, Palladium¹⁰³ and Iridium¹⁹².

The source of radiation may be temporary or permanently implanted in the tumor. Temporary implants are removed after a certain period of time, and refer to a High-Dose Radiation Rate. Permanent implants are left in the tissues and the radiation decays over time, and refer to a Low-Dose Radiation Rate. The rationale for using an implant is that it can deliver the radiation to a small area while increasingly sparing the surrounding normal tissues which are not irradiated. Interstitial brachytherapy refers to the placement of sources into tissues, while intracavitary brachytherapy refers to the placement of sources into a cavity, such as the uterus. Brachytherapy is often used in the treatment of cancers of the cervix, uterus, prostate, breast, lung, head and neck area. It can, however, be used almost anywhere in the body when appropriate.

### Advantages of Brachytherapy and its Constraints

During the last five years brachytherapy has become the most favored treatment for specific types of cancer, most typically, prostate cancer. It has been reported that in 2003, more than 50,000 patients were treated with prostate brachytherapy in the USA and 4,000 in Europe. These numbers show that, for the first time, more patients were treated with permanent seed prostate brachytherapy than by surgery. The most important advantages of permanent seed brachytherapy for localized cancers compared to standard surgery include:
1. Short period of hospitalization, typically a few hours treatment within a period of a few days of hospitalization;
2. Reduced risks related to surgical operation;
3. Delivery of a precise radiation dose to the infected cancerous cells, while sparing the surrounding healthy tissues;
4. Limited side effects, thus preserving quality of life for the patient;
5. Combination of other techniques normally used in operations such as ultrasonic probes for precise placement of seeds in the infected tissues.

There are two problems associated with the use of radiation in cancer treatment. The first is the desire to keep the level of radiation applied as low as possible. The second is that the DNA, whose mass is merely 0.25% the mass of the entire cell, has been identified as the critical target for killing the cell with radiation. Therefore, the probability for the quanta of energy emitted by photons (emitted from the radiation source) to interact and be adsorbed at the DNA target site is extremely small. The use of Auger electrons addresses both problems.

The Auger effect occurs when an incident radiation, a photon or an electron, removes an electron from an inner shell of the atom. The vacancy created can be filled by an outer shell electron from the same atom, in which case the electron moves to a lower energy state, and the energy associated with the transition is the difference in orbital energies. This energy must be released in some fashion. In some cases this energy is emitted as an x-ray and in other it is imparted to a second outer shell electron, which then is ejected from the atom. The characteristic energy of this ejected electron is the energy obtained in the electron transition minus the binding energy of the ejected electron.

The Auger process involves three steps:

1. Excitation or ionization of the metal atom causing emission of an electron;

2. One electron dropping down to fill the vacancy created in the first step; and

3. The emission of an Auger electron triggered by the energy released in the second step.

The potential of the Auger electrons to effectively damage the DNA of the malignant cell depends on the localization of the metal atom (the Auger emitter), which should be as close as possible to the DNA or within the range of the Auger electrons in the cell. The electron range depends on their energy, and it is about the size of the DNA diameter namely, 20 nm.

A detailed description of cancer therapy based on the Auger effect may be found in, for example, WO 03/063757, incorporated herein by reference.

One objective of the present work is to develop a reliable method to introduce a large number of Auger electron-emitting atoms into a diseased cell of a subject suffering from an immune-related disorder, or a malignant or non-malignant proliferative disorder. Thus, the invention provides a most efficient system for cancer and rheumatoid arthritis therapy. The solution found by the present inventors is the use of liposomes in order to introduce heavy atoms into the cell, particularly gold and platinum in various chemical states. Although similar solutions have been described in the art, the present inventor has developed heavy metal-containing nano-liposome particles, particularly liposomes bearing gold and platinum, which are superior in their stability and in their ability to effectively deliver heavy atoms into target cells, as well as in their reduced immunogenicity. The efficient delivery of heavy atoms into diseased cells, particularly cancer cells, results in most efficient killing of such cells either upon irradiation, or upon induction of biomechanisms which directly or indirectly trigger cell death.

As described by the following examples, the inventors have surprisingly found that efficient elimination of diseased cells of a subject suffering from an immune-related disorder, may also occur by using only the nano-particles of the invention with no irradiation. Therefore, an additional object of the invention is to provide methods, compositions and kits using the nano-particles of the invention for the treatment of immune-related disorders. More particularly, the methods and compositions of the invention are intended for the treatment of malignant and non-malignant proliferative disorders, such as cancer and/or inflammatory disorders such as arthritis. It should be appreciated that the main advantage of using the nano-particles of the invention without irradiation is avoiding of irradiation side effects.

Arthritis has been categorized into as many as 12 types, the most common being osteoarthritis and rheumatoid arthritis. Rheumatoid arthritis is a chronic condition that can result in weakness, loss of mobility, and eventual destruction and deformity of the joints. Osteoarthritis (also called degenerative joint disease) usually affects people after middle age and is characterized by gradual loss of cartilage of the joints. It can result in joint disfigurement and restricted joint mobility.

The exact mechanism by which gold salts work in the treatment of inflammatory arthritis is not well understood. Nonetheless, it has been shown to decrease the inflammation of the joint lining, and in this way prevent the destruction of bone and cartilage, as well as the painful symptoms to the patient.

Thus, it is a main object of the present invention to provide novel heavy metal-containing nano-liposomes particles and their method of preparation, which includes novel nano-liposomes and their preparation and novel stable heavy metal complexes and their preparation. Further, the present invention also provides the use of the heavy metal-containing nano-liposome particles in medical treatment of immune-related disorders, particularly cancer and arthritis, either for brachytherapy in combination with a radioactive source (seed) and irradiation, or alternatively, the use of these heavy metal-containing nano-liposome particles by themselves for the treatment of immune-related disorders such as cancer and inflammation.

The disclosed heavy metal-containing liposomes can also be used for other biomedical applications, such as the delivery of heavy metal ions to organic tissues. Of particular interest, the developed nano-liposomes may be used for the delivery of iron ions to the circulatory system through the digestive system.

Other uses and objects of the invention will become clear as the description proceeds.

### SUMMARY OF THE INVENTION

In a first aspect, the present invention provides a method of preparing restructured complex phospholipids by enzymatic transphosphatidylation, wherein the enzyme and the alcohol or polyol are immobilized both on a water-insoluble support and the phospholipids substrate is solubilized in organic solvent, said method comprising the steps of:

(a) Solubilizing phospholipase D (PLD) enzyme in a suitable buffer solution, preferably acetate buffer pH 6.5;
(b) Immobilizing the enzyme on an insoluble matrix;
(c) Mixing the immobilized enzyme with an alcohol or polyol having a molecular weight of from 100 to 20,000 Daltons, wherein said polyol is preferably polyethylene glycol (PEG) or polyglycerol;
(d) Adding the mixture comprised of the matrix support loaded with PLD and alcohol or polyol substrate, such as PEG, prepared in step (c) to an organic solution containing phosphatidyl choline (PC) and mixing for a suitable period of time to allow the transphosphatidylation reaction to occur;
(e) Separating the organic phase from the inorganic phase;
(f) Washing the organic phase with distilled water to remove unreacted alcohol or polyol; and
(g) Optionally further washing the organic phase with an aqueous solution of a suitable metal ion chelator, preferably EDTA, to remove the calcium ions; said method yielding a transphosphatidylated end product comprising a polyol-phospholipid or polyol-diphospholipid. In particular, said end product is a mixture of pegylated phospholipid (PEG-PL) and pegylated di-phospholipid (PEG-DPL), optionally containing unreacted PL.

Thus, the present invention provides phospholipids and di-phospholipids having a modified alcohol moiety prepared by the method as described above.

In another aspect, the present invention provides a method of preparing a stable, water soluble heavy metal complex, said method comprising the steps of:

(a) Dissolving in water an ionic heavy metal species, to obtain a transparent gold solution;
(b) Adding an organic ligand to the heavy metal solution of step (a), wherein said ligand is capable of binding heavy metal and forming a heavy metal complex which is water insoluble or water soluble at pH values of below 6, preferably below 4.5;
(c) Adjusting the pH of the mixture of step (b) to a value of at least 4.5 with a suitable base, preferably sodium hydroxide solution, yielding a heavy metal complex of high solubility in water solutions of pH 4.0 or higher.

Where said heavy metal species is gold, preferred forms to be used in the above-described method are HAuCl₄ and MAuCl₄, wherein M represents an alkali metal cation. Where said heavy metal species is platinum, the preferred form to be used in the above-described method is K₂PtCl₄. Where said heavy metal species is iron, preferred forms to be used in the above-described method are FeCl₂, FeSO₄ and FeCl₃.

The present invention also provides a method of preparing a stable, water soluble heavy metal complex starting from an organic solution, said heavy metal being e.g. gold, platinum or iron, said method comprising the steps of:

(a) Dissolving in an organic solvent an ionic heavy metal species, which may be but is not limited to gold, such as AuCl₃, AuBr₃, or AuF₃, and a surface active ingredient such as quaternary ammonium salt, to obtain a transparent heavy metal solution;
(b) Adding an organic ligand to the heavy metal solution of step (a), wherein said ligand is capable of binding heavy metal and forming a heavy metal complex;
(c) Evaporating the organic solvent of step (b) and dissolving the heavy metal complex residue in water;
(d) Adjusting the pH of the mixture of step (c) to a value of at least 4.5 with a suitable base, preferably sodium hydroxide solution, yielding a heavy metal complex of high solubility in water solutions of pH 4.0 or higher.

In one specific embodiment of this method of the invention, said organic solvent is selected from the group comprised of toluene, dichloromethane, dialkylethers and tetrahydrofurane.

In one specific embodiment of the method of preparing a stable, water soluble heavy metal complex, starting from an organic or aqueous solution, said organic ligand is selected from the group comprised of N-acyl cysteine, preferably N-acetyl cysteine, amino acids in particular cysteine and methionine, glutathione, amino thiols, thio-carboxylic acids, diamines, and any organic ligand capable of binding heavy metal atoms and forming a water-soluble heavy metal complex.

Thus the stable, water-soluble heavy metal complex, produced by the methods described above, is also part of the presently claimed invention.

In an even further aspect, the present invention provides a water-soluble heavy metal complex comprising an ionic heavy metal species, such as HAuCl₄ and MAuCl₄, wherein M designates an alkali metal cation, and an organic ligand, said complex being soluble and resistant to chemical reduction by mild reducing agents, such as amines, thiols and citrate, in aqueous solutions having pH values higher than 4.0, wherein the molar ratio of said heavy metal ionic species to said organic ligand is preferably from at least 0.5:1 to 1:4 or any higher ratios.

In an additional aspect the present invention provides a method of preparing a stable, water-soluble heavy metal complex in the presence of polar lipids or any other mild reducing agents such as citrate, said method comprising the steps of:

(a) Dissolving an ionic heavy metal species, such as HAuCl₄ and MAuC₄, wherein M designates an alkali metal cation, preferably gold trichloride in water, to obtain a transparent ionic gold solution;
(b) Adding a ligand capable of binding the heavy metal ions, preferably N-acyl cysteine, to produce a heavy metal complex which is water insoluble at pH values below 6;
(c) Adjusting the pH of the mixture of step (b) to a value of at least 4.5, with a suitable base, preferably sodium hydroxide solution, yielding a heavy metal complex of high solubility in water solutions of pH 4.0 or higher;
wherein said heavy metal complex is water soluble at pH values in the range of between 5-9, preferably at physiological pH. A stable, water-soluble heavy metal complex produced by this method is also contemplated by the present invention.

In one particular embodiment of this method, said reducing factors are polar lipids or mild reducing factors, preferably citrate, amines, and thiols.

In an even further aspect, the present invention provides a water-soluble heavy metal complex comprising an ionic heavy metal species and one of N-acyl cysteine or cysteine, said complex being water-soluble and resistant to chemical reduction by mild chemical reducing agents in aqueous solutions having pH higher than 4.0, and particularly at physiological pH values, wherein the molar ratio of said heavy metal ionic species to said N-acyl cysteine is from 0.5:1 to 1:4 or higher.

The heavy metals to be used in producing the heavy metal complex generated by any of the methods described in the invention are selected from the group comprised of gold, platinum, iron, silver, copper, nickel, palladium, iridium, titanium and any other heavy metal of therapeutic use.

A further yet aspect of the present invention is a method of preparing nano-liposomes comprising ionic heavy metal or atomic metal, said method comprising the steps of:

(a) Mixing a heavy metal complex in accordance with the invention, or prepared by the methods of the invention, with a liposome-forming surface-active material;
(b) Homogenizing the mixture of step (a), obtaining large multi-lamellar vesicles;
(c) Optionally applying freeze-thaw cycles to the large multi-lamellar vesicles, and generating smaller vesicles;
(d) Sizing the mixture of step (c) by multiple extrusions, sonication or by using a microfluidizer, to yield heavy metal-containing nano-liposomes, in the size of from 15 to 150 nm.

In one specific embodiment of said method, said liposome-forming surface-active material is selected from the group comprised of phospholipids (e.g. phosphatidyl choline, phosphatidyl ethanolamine, phosphatidyl glycerol, phosphatidyl inositol, phosphatidyl serine, PEG-phospholipids, and ether phospholipids), ceramides, sphingomyelins, and cholesterol.

Another further aspect of the present invention is a method of preparing nano-liposomes comprising ionic heavy metal or heavy metal atoms (or, heavy metal-containing nano-liposomes), said method comprising the steps of:

(a) Dissolving cholesterol and a mixture of phospholipids, preferably PEG-PL and PEG-DPL, in an organic solvent;
(b) Evaporating the organic solvent under suitable conditions, preferably under vacuum at 50° C., to yield a lipid film;
(c) Drying the lipid film obtained in. step (b) under suitable conditions to remove residual organic solvent and/or water;
(d) Mixing a heavy metal complex in accordance with the invention as described above, or obtained as a final product of one of the methods described above, with the lipid film of step (c) and allowing hydration of said lipid film;
(e) Homogenizing the mixture of step (d), obtaining large multi-lamellar vesicles;
(f) Optionally applying freeze-thaw cycles to the large multi-lamellar vesicles, generating smaller vesicles;
(g) Sizing the mixture of step (f) by multiple extrusions, sonication or by using a microfluidizer, to yield heavy metal-containing nano-liposomes, in the size of from 15 to 150 nm.

Most importantly, said PEG-PL and PEG-DPL mixture may also be obtained by the method of preparing said mixture as described in the present invention.

The present invention thus also provides the heavy metal-containing nano-liposome particles prepared by the method described herein, wherein said heavy metal exists in its ionic or atomic state.

One additional important aspect of the present invention is to provide a heavy metals-containing nano-liposome particle comprising a complex of water-soluble heavy metal ions or heavy metal atoms, particularly gold, platinum or iron, and an organic ligand, entrapped in an organic layer comprising pegylated phospholipids and di-phospholipids or any liposome-forming surface-active ingredient, wherein said heavy metal complex is stable at physiological pH, or in a living cell environment in in vivo or in vitro conditions.

Even further, the present invention provides a pharmaceutical composition comprising the heavy metal-containing, such as gold, platinum or iron, nano-liposome particles described above, or prepared by the above-described methods. This composition optionally further comprises at least one additional therapeutic agent and optionally further comprises pharmaceutically acceptable additives, carriers, buffers, stabilizers and/or excipients. Said pharmaceutical composition may be for use in medical therapy, particularly in the treatment of an immune-related disorder such as malignant and non-malignant proliferative disorder, inflammatory disease, metal deficiency, autoimmune diseases and metal deficiency-related physiological disorders.

According to one embodiment, the pharmaceutical composition of the invention is intended for the treatment of a malignant proliferative disorder such as a solid tumor selected from the group consisting of carcinoma, sarcoma and melanoma.

The pharmaceutical composition may also be used for the treatment of an inflammatory disease such as rheumatoid arthritis and osteoarthritis, particularly rheumatoid arthritis.

Alternatively and additionally, the pharmaceutical composition of the invention is intended for the delivery of metals to the circulatory system or to other organs, to treat a metal deficiency, or for diagnostic applications.

The invention further provides for the use of the gold- or platinum-containing nano-particles described herein, in the preparation of a pharmaceutical composition for the treatment of an immune-related disorder. More particularly, such immune-related disorder may be a malignant or non-malignant proliferative disorder, particularly, a solid tumor or alternatively, an inflammatory disease such as rheumatoid arthritis orosteoarthritis.

Still further, the invention provides a kit for the treatment of a patient suffering from an immune-related disorder. The kit of the invention preferably comprises the following components: (a) heavy metal-containing nano-liposome particles suspended in an aqueous system, as defined by the invention, or a composition comprising such particles; (b) means for administering the heavy metal-containing nano-liposome particles into the diseased cell or tissue of a patient in need of such therapy; and (c) instructions for use.

According to one embodiment, the kit of the invention may be particularly used for the treatment of malignant and non-malignant proliferative disorder, particularly a solid tumor such as a carcinoma, sarcoma and melanoma.

According to another embodiment, the kit of the invention may be used for the treatment of inflammatory disease such as rheumatoid arthritis or osteoarthritis.

The invention further provides a method for the treatment of an immune-related disorder. The method of the invention involves administering to a subject in need thereof a therapeutically effective amount of the gold- or platinum nano liposome particles of the invention or a pharmaceutical composition comprising these particles. It should be appreciated that the method of the invention may optionally use the kit as defined by the invention.

According to one embodiment, administration of the active compound by the method of the invention may involve intravenous, intraperitoneal, intra-tumor, intragastric, or topical injection, or orally, or any combination thereof.

It should be noted that the method of the invention may be particularly applicable for the treatment of immune-related disorder such as malignant and non-malignant proliferative disorder, inflammatory disease, metal deficiency or an autoimmune disease.

According to one embodiment, the method of the invention may be used for the treatment of a malignant proliferative disorder, particularly, a solid tumor such as carcinoma, sarcoma and melanoma.

According to another embodiment, the method of the invention may be used for the treatment of inflammatory disease such as rheumatoid arthritis or osteoarthritis.

In a much further aspect, the invention relates to a kit for use in brachytherapy of an immune-related disorder, specifically, in the therapy of a malignant proliferative disorder, for example, a solid tumor such as carcinoma, sarcoma and melanoma. The kit of the invention preferably comprises the following components: (a) heavy metal-containing nano-liposome particles in aqueous system, as defined by the invention, or a composition comprising these particles; (b) implantable seeds of a radioactive source, wherein said source is selected from the group consisting of Thulium (¹⁷⁰Tm), ¹⁰³Pd, ¹⁴⁵Sm, ¹²⁵I, a mixture of ¹²⁵I and ¹²⁷I, ²³⁴Th, ^{93m}Nb, ¹⁴⁰Ba, ¹⁹⁵Au, ¹⁴⁴Ce, ^{125m}Te, ^{95m}Tc, ²⁴⁵Am, ²⁵³Eu, ¹⁸³Re, ¹⁸⁵W, ¹⁵⁹Dy, ^{127m}Te, ¹⁶⁹Yb, ¹⁰⁵Ag, ^{119m}Sn, ¹⁷¹Tm, ¹⁴⁵Pm, ¹⁵³Gd, ¹³³Ba, ¹⁷⁴Ln, ¹⁶³Tm, ¹⁴⁷Eu, ¹⁷⁵Hf and ^{97m}Tc, wherein said radioactive source promotes an Auger effect in combination with heavy metal; (c) means for delivering said heavy metal-containing nano-liposome particles, such as gold-containing or platinum-containing nano-liposomes, to a patient in need of brachytherapy; (d) means for delivering said implantable seeds to said patient and (e) instructions for using the kit.

Still further, the invention provides a method of brachytherapy treatment of a malignant proliferative disorder, comprising the step of administering to a subject in need thereof a therapeutically effective amount of (a) the heavy metal-containing nano-liposome particles in aqueous system, as defined by the invention, or alternatively, a composition comprising thereof; and (b) implantable seeds of a radioactive source. Such radioactive source may be selected from the group consisting of Thulium (170Tm), ¹⁰³Pd, ¹⁴⁵Sm, ¹²⁵I, a mixture of ¹²⁵I and ¹²⁷I, ²³⁴Th, ^{93m}Nb, ¹⁴⁰Ba, ¹⁹⁵Au, ¹⁴⁴Ce, ^{125m}Te, ^{95m}Tc, ²⁴⁵Am, ²⁵³Eu, ¹⁸³Re, ¹⁸⁵W, ¹⁵⁹Dy, ^{127m}Te, ¹⁶⁹Yb, ¹⁰⁵Ag, ^{119m}Sn, ¹⁷¹Tm, ¹⁴⁵Pm, ¹⁵³Gd, ¹³³Ba, ¹⁷⁴Ln, ¹⁶³Tm, ¹⁴⁷Eu, ¹⁷⁵Hf and ^{97m}Tc, wherein said radioactive source promotes an Auger effect in combination with heavy metal atoms, such as gold or platinum. It should be noted that this method preferably uses the brachytherapy kit as defined by the invention.

### BRIEF DESCRIPTION OF THE FIGURE

FIG. 1: Enzymatic transphosphatidylation for the production of pegylated phospholipids and di-phospholipids.

### DETAILED DESCRIPTION OF THE INVENTION

The present inventors have developed novel liposomes for the targeted release and delivery of drugs to be used in the treatment of cancer and inflammatory diseases, particularly arthritis, as well as for the treatment of metal deficiency-related physiological disorders.

Thus, the present invention describes novel liposomes, and methods for their preparation, novel heavy metal complexes stable at physiological pH and methods for their production, and finally the heavy metal-containing liposome particles and their method of production, as well as therapeutical compositions containing them and their uses in medical treatment.

The heavy metal-containing nano-liposomes presented in the invention are produced by novel preparation methods as described below, particularly in Example 2 (which describes the preparation of the liposomes), Example 3 (which describes the preparation of stable gold complexes), Example 4 (which describes the preparation of the gold-containing nano-liposomes per se), and Examples 12 and 13 which describe the preparation of platinum- and iron-containing nano-liposomes, respectively. Said heavy metal-containing nano-liposomes contain highly loaded nano-size particles of heavy metals in their atomic or ionic state. The main use of the heavy metal-containing nano-liposome particles of the invention, as demonstrated in the Examples, is in the control and targeted release of gold or platinum atoms or other metals into diseased, particularly malignant cells for various diagnostic and mainly therapeutic purposes. Similarly, iron-containing nano-liposomes are used, for example, in the delivery of iron to the circulatory system for treatment of patients who suffer from iron deficiency. The heavy metal-containing nano-liposomes of the invention therefore are used as therapeutic compositions in methods and kits for treating immune-related disorders such as proliferative disorders as cancer, or inflammatory disorders such as arthritis. Alternatively and additionally, these particles may be used for the brachytherapy treatment of cancerous disorders in combination with irradiation for targeting and enhancing selective irradiation of cancer cells using a radioactive source for the purpose of their destruction.

The liposomes provided herein are prepared using enzymatically-produced restructured phospholipids, and have shown higher stability to hydrolytic factors as well as a higher degree of tolerance when exposed to macrophages in the circulation system. This latter property was described for liposomes

[Stearne, L. E. T. (2002) Biochimica. et Biophysica Acta 1561: 91-97]. Nonetheless, the liposomes prepared by the methods of the present invention have an even higher tolerance, or survival, when exposed to macrophages, since the PEG-di-phospholipids described herein create a net-like structure on the surface of the liposome membrane which hinders them (the liposomes) from being engulfed and/or cytophaged. This is an advantage over the liposomes described in the prior art, which are usually produced employing standard methods using various phospholipids, as e.g. phosphatidyl choline, combined with cholesterol and PEG-mono-phospholipids (PEG-PL) and other surface-active ingredients, and usually are degraded more easily when used systemically.

The present invention describes mainly enzymatic preparation methods for producing a mixture of modified phospholipids (PL) (by enzymatic trans-phosphatidylation). It is to be understood that this type of PL composition can be chemically synthesized as well.

In the past, the preparation of stabilized heavy metal-containing liposome particles has met many obstacles, including: (i) forming stabilized heavy metal nano-particles of 2-6 nm in size and preventing their aggregation; (ii) stabilizing heavy metal ions prior to their reduction for the preparation of heavy metal nano-particles; (iii) inserting a critical number of water-soluble heavy metal complexes or heavy metal particles of nano-particle size into stabilized liposomes that can be injected into the body's circulation system; and (iv) releasing them specifically into the affected sites in the body.

The present inventor has found a solution to these obstacles, by producing stable heavy metal complexes, which are soluble at physiological pH, and which, when provided in the form of water-soluble heavy metal complexes or heavy metal-containing nano-liposome particles, with or without a radioactive source, were able to deliver a significantly higher number of heavy metal ions into the cell. As shown in Example 7, typically, more than 1×10⁸ gold atoms are delivered per cell.

Thus, essentially, the present invention encompasses a few aspects, which may be summarized as follows: the preparation of the nano-liposomes, the preparation of stable heavy metal complexes, the preparation of the heavy metal-containing nano-liposomes, compositions containing them, and their use in medical treatment, specifically for cancer and inflammation. These aspects shall be described in further detail below, and are exemplified in the Examples section.

The preparation of the nano-liposomes involves the enzymatic trans-phosphatidylation of phospholipids with an alcohol or polyol. The preferred enzyme is phospholipase D (PLD), such as PLD from Streptomyces (Asahi Chemical Industry Co. Japan), Actinomadura and Nocardiopsis (Meito Sangyo, Japan), from genetically modified E. coli, or from cabbage (Sigma-Aldrich, Israel). The phospholipid (PL) used may be any one of phosphatidylcholine (or lecithin), phosphatidylglycerol, phosphatidylinositol, phosphatidylserine, and phosphatidylethanolamine, from animal, marine or plant source. Preferred PL is egg lecithin or soy lecithin. The alcohol or polyol of choice in the present invention was polyethylene glycol (PEG), which may be of a molecular weight of between 100 Dalton to 20,000 Daltons, preferably from 100 to 10,000 Daltons, and even more preferably from 200 to 1000 Daltons. Other alcohols or polyols which may alternatively be employed are polyglycerol and other polyglycols.

In the reaction, the enzyme is solubilized in a suitable buffer solution, such as citrate or Tris, or preferably acetate buffer at pH6.5, and is immobilized on a water-insoluble matrix support such as Celite, silica gel, ion exchange resin or any suitable adsorbent matrix. The immobilized enzyme is then mixed with the alcohol- or polyol-saturated aqueous solutions, preferably PEG, to give an insoluble matrix loaded with PLD and PEG, and afterwards mixed with the phospholipid, preferably PC, dissolved in an organic solvent (e.g. ether, chloroform, ethyl acetate and dichloromethane). The immobilized enzyme is then filtered off. After evaporation of the solvent, a product is obtained which contains modified phospholipids, particularly PEG-phospholipids and PEG-diphospholipids. It should be noted that the first step may be as described above, mixing the enzyme with the matrix, and then adding the alcohol- or polyol-saturated aqueous solution or, alternatively, the enzyme may be mixed with the alcohol- or polyol-saturated aqueous solution, and then add the matrix.

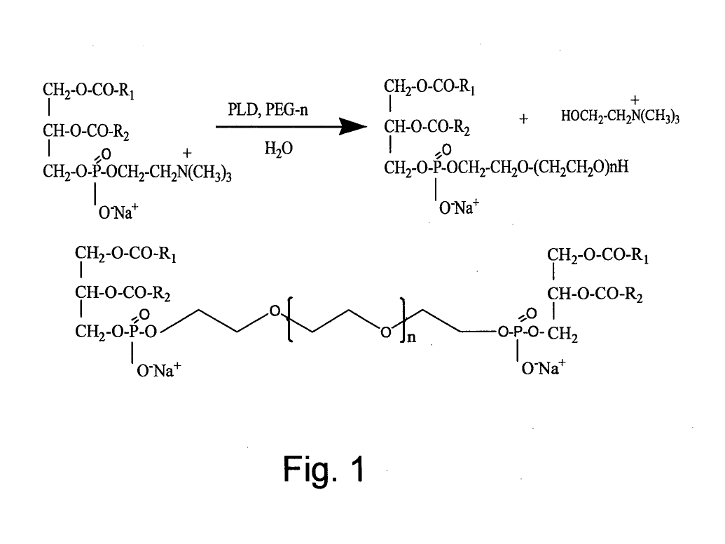

The detailed preparation of modified phospholipids is described in Examples 1 and 2 below. Pegylated phospholipids (PEG-PL) of the chemical structure as described below in Formula 1 are well documented in the literature. One of their important features is their long lasting period in circulation (in the blood system) when used in the formation of liposomes. PEG-phospholipids are synthesized enzymatically with PLD or chemically, using activated polyethylene glycol (PEG) derivatives. U.S. Pat. No. 5,153,000 describes a method for producing PEG-di-phospholipids (PEG-DPL) (Formula 2) using PLD in water system (FIG. 1). In the present study, modified preparation methods for producing a mixture of PEG-PL and PEG-di-PL are introduced. Particularly, the use of an organic system is described in Example 2, which constitutes an important feature of the invention. The advantage of this process is that it yields highly pegylated phospholipids, which are advantageous in the preparation of liposomes for medical use, as such liposomes better mask the foreign agents contained or entrapped therein, thus having reduced immunogenicity.

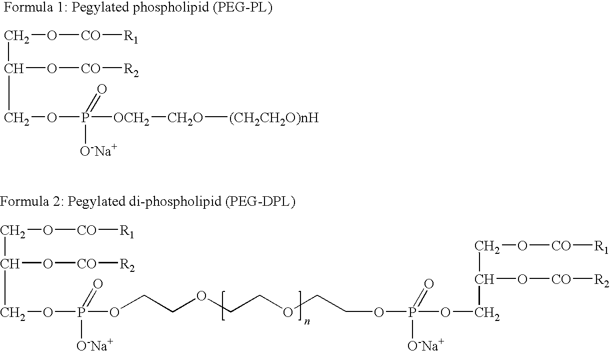

Once the transphosphatidylation reaction is complete, the end product is polyol-phospholipids and polyol di-phospholipids, which are exemplified herein by PEG-PL and PEG-DPL.

The necessity to generate metal-containing nano-liposome particles which make use of the Auger effect, and their uses in therapeutic methods has met with two main difficulties. The first is related to the placement of the element undergoing the Auger effect in close proximity to the DNA. The second is related to the photons energy emitted from the source, which must be a little higher than the k edge energy of the Auger electrons emitter. This is required so that the occurrence of the photoelectric effect is capable of removing the electron from the atom k shell, and start an avalanche of electrons, followed by Auger electrons emission. The heavier the atom the more electron shells are available for electron emission, and therefore more Auger electrons are emitted. Gold and platinum are within the heaviest atoms and thus are very good candidates for the emission of a large number of Auger electrons. The radiation source in the present system emits photons of energy 84 keV and the gold k shell energy is 80.7 keV.

Thus, in the present invention the preparation of heavy metal-containing nano-liposome particles was preceded by the preparation of stable, water-soluble heavy metal complexes. The metal used in the preparation of said complexes may be any one of gold, platinum, silver, iron, copper, nickel, palladium, iridium, titanium, and other heavy metals of therapeutic use. Other heavy metals or heavy metal complexes of therapeutic use include, but are not limited to, cisplatin, carboplatin, cerium, tungsten, strontium, lanthanum and ruthenium. Preferably said metal is gold, platinum or iron.

The preparation of a stable heavy metal complex comprises the steps of: (a) dissolving in water an ionic heavy metal species, to obtain a heavy metal solution; (b) adding an organic ligand to said heavy metal solution, wherein said ligand is capable of binding the heavy metal and forming a complex, which is water soluble or water insoluble; (c) adjusting the pH of said mixture with a suitable base, to a value in which said heavy metal complex achieves high solubility in water. The base of preference used is sodium hydroxide solution or other alkali metal hydroxide, but other bases are also suitable including amine derivatives, such as ammonia, secondary and tertiary amines. Said organic ligand is selected from the group comprised of N-acetyl cysteine, and amino acids, in particular cysteine and methionine, glutathione, thio-carboxylic acids, ammonia and amines, amino thiols, diamines, and any organic ligand capable of binding heavy metal and preferably, but not necessarily, forming a water-soluble heavy metal complex.

In particular, when preparing a stable, water soluble metal complex with gold, an ionic gold species selected from HAuCl₄ and MAuCl₄ (where M represents an alkali metal cation, for example sodium or potassium cations) dissolved in water is mixed with an organic ligand which binds to the gold atoms, forming a water-insoluble gold complex at pH values below from about 6 to about 4.5, but water soluble at pH values of 4 or higher, respectively. The solubility of the gold ionic species/ligand complex (and any heavy metal complex) is affected by the nature of the ligand, particularly by its pKa. Thus, the solubility is adjusted by selecting an organic ligand with a pKa suitable for water solubility at a certain pH. Said organic ligand is selected from the group comprised of N-acetyl cysteine, and amino acids, in particular cysteine and methionine, glutathione, thio-carboxylic acids, ammonia and amines, amino thiols, diamines, and any organic ligand capable of binding gold and forming a water-soluble gold complex at pH values of from about 4.5 to about 6. The ideal organic ligand is that which can form a water-soluble gold complex at a pH of physiological range, or at a pH between 5 and 9, preferably between 7.0 and 7.5. One specific example of such organic ligand is N-acetyl-cysteine, which typically yield complexes that are water insoluble at pH below 4. The preparation of water-soluble stable gold complex in accordance with the invention is particularly described in Example 3.

The formation of a precipitate upon the addition of an organic ligand solution to the metal solution is dependent on the type of metal and the type of ligand. For example, gold ions precipitate in combination with N-acetyl cysteine or cysteine. In contrast, platinum ions form a precipitate with cysteine, but not with N-acyl cysteine. Most importantly, any of these complexes is water soluble at physiological pH.

Preferred water soluble platinum complexes were generated with ionic platinum metal species and ammonia.

Essentially, said stable, water soluble metal complex with gold, platinum or iron, as described in the invention is soluble at pHs 4, 4.5, 5, 5.5, 6, 6.5, 7, 7.2, 7.4, 7.5, 7.6, 8.0, 8.5, 9 and above.

One important property is that the heavy metal complex should be water soluble in the presence of polar lipids or any other reducing factors (or agents), particularly polar lipids or reducing factors typically present under physiological conditions, e.g. citrate, amine derivatives and thiols.

The heavy metal complex modified with N-acyl cysteine may also be synthesized in an organic system, starting e.g. with AuCl₃ (when the heavy metal is gold) solubilized in an organic solvent such as toluene. When this mixture is treated with N-acyl cysteine, it yields the same complex as described above with the aqueous system. After evaporating the organic solvent, the complex can be dissolved in water upon pH adjustment with an alkaline solution, such as sodium hydroxide and other alkali metal hydroxides, or with other bases such as amine derivatives.

By the term “gold ionic species” as used herein is meant any substance or chemical, entity that contains or can generate (and thus is a precursor of) gold ions. Thus, for example HAuCl₄ is a precursor ionic species which yields [AuCl₃]⁻ ions when reacted with N-acyl cysteine.

Heavy metal-containing nano-liposome particles were essentially prepared by mixing the transparent aqueous solution of the stable, water-soluble heavy metal complex of the invention with a dried mixture of PEG-PL and PEG-DPL and cholesterol. Said heavy metal solution+lipid mixture was homogenized to obtain large multi-lamellar vesicles, which were optionally treated with freeze-thaw cycles in order to obtain smaller vesicles. These smaller vesicles were further sized through extrusion or using a microfluidizer, to yield heavy metal-containing nano-liposomes of sizes ranging from 10 to 150 nm, preferably from 15 nm to 70 nm, particularly 20 to 50 nm, and more preferably from 20 to 40 nm.

The heavy metal-containing nano-liposomes may also be prepared by mixing the water-soluble heavy metal complexes with phospholipids prepared in a water-based system, such as that described in U.S. Pat. No. 5,153,000.

As mentioned before, these heavy metal-containing nano-liposome particles are to be used therapeutically, either in brachytherapy for solid tumors, or topically in the treatment of immune-related disorders such as inflammatory disorders as well as malignant or non-malignant proliferative disorders that may be solid or non-solid tumors. The particles may be used per se, or as an active agent comprised in a pharmaceutical composition. It should be appreciated that the heavy metal-containing nano-liposome particles of the invention may also be used for targeted delivery of heavy metals, or drugs comprising heavy metals, and therefore may also provide a platform for combination therapy.

Interestingly, the heavy metal containing liposome particles, particularly gold and platinum particles, may also be used alone (i.e., without combination with brachytherapy) in the treatment of cancer, and particularly in the treatment of solid tumors. As shown in Example 10 below, treatment of mice bearing solid tumors with gold-containing liposome particles resulted in the disappearance and cure of said tumors.

The preparation of pharmaceutical compositions is well known in the art and has been described in many articles and textbooks, see e.g., Remington's Pharmaceutical Sciences, Gennaro A. R. ed., Mack Publishing Co., Easton, Pa., 1990, and especially pp. 1521-1712 therein.

Compositions and formulations for parenteral, intra-peritoneal, intrathecal, intragastric, intraventricular, intravenous and oral administration may include sterile aqueous solutions which may also contain buffers, diluents and other suitable additives such as, but not limited to, penetration enhancers, carrier compounds and other pharmaceutically acceptable carriers or excipients.

When used in medical therapy, and administered systemically, the heavy metal-containing nano-liposome particles prepared according to the above-described procedures have a high degree of tolerance in the circulatory system towards macrophages on one hand, and on the other hand are degradable by phospholipase A2 and other hydrolytic enzymes.

As demonstrated in the examples below, the gold-containing nano-liposome particles described herein are preferably delivered by intravenous, intraperitoneal or topical injection, and reach a critical number of gold atoms in the target cells of typically higher than 1×10⁸ atoms per cell. Said target cell may be a diseased cell of a subject suffering from an immune-related disorder, for example, a tumor cell, an infected cell, or a cell from inflamed tissue.

Similarly, iron-containing nano-liposome particles are preferably delivered intragastrically or orally, or any form that facilitates the particles of reaching the circulation.

Thus, the heavy metal-containing nanoliposomes of the invention may be delivered per se, comprised in a pharmaceutical composition or formulation, or also comprised in a nutraceutical composition.

When applied for brachytherapy, the heavy metal-containing nano-liposome particles are used in order to insert a heavy element, particularly gold or platinum, inside the tumor cells, and thus in closer proximity to the DNA. The radiation source activates said heavy element and causes the emission of Auger electrons therefrom, increasing the damage caused to the DNA (this increase is in comparison to using the radiation source alone for inducing DNA damage). Usually the radiation source produces photons, whose energy is above the binding energy of the electron in the K-shell, or in the L-shell, of the heavy element. K-shell energy values of the elements are known in the art [C. M. Lederer and V. S. Shirley (1978) “Table of Isotopes”, Wiley and Sons].

This is the Auger effect described above in the Background section. Preferred heavy elements used in the present invention are gold, platinum, silver, copper, nickel, palladium, and iridium.

The preferred radiation source to be used in the present invention is an implanted one. Implanted radiation sources comprise a radioactive isotope packed within a casing, said casing being usually in the form of a closed, cylindrically shaped, canister. Typical dimensions for such canisters, which are also known as seeds, are around 0.45 mm in diameter and between 0.5 and 1.0 cm in length. The canister is preferably made of any one of titanium, stainless steel, vanadium, inert bioceramics, glass and porcelain. The source is thus prepared by loading the canister with the selected radioisotope and sealing the canister, by laser welding or any other method known in the art, such as laser welding, electron beam welding, crimp welding, gas tungsten arc welding, gas metal arc welding, flux cored arc welding, shielded metal arc welding or submerged arc welding. Implantable radiation sources (brachytherapy seeds) and methods for producing the same have been described in the literature [e.g. U.S. Pat. No. 6,132,359; Chen et al. (2001) Med. Phys. 28, p. 86-96].

Implantation of brachytherapy seeds has been described, e.g., in U.S. Pat. No. 6,036,632, U.S. Pat. No. 6,267,718, and U.S. Pat. No. 6,311,084. Essentially, the seeds are implanted such that the most optimal radiation levels reach the target tissue, according to the shape of the patient's tumor. In prostate cancer for example, the seeds are loaded into the cannula of a needle-like insertion device.

Thus, the radiation source for the present invention is usually prepared by loading small tubes, preferably made of titanium, with at least one radioisotope selected from the group consisting of Thulium (170Tm), ¹⁰³Pd, ¹⁴⁵Sm, ¹²⁵I, a mixture of ¹²⁵I and ¹²⁷I, ²³⁴Th, ^{93m}Nb, ¹⁴⁰Ba, ¹⁹⁵Au, ¹⁴⁴Ce, ^{125m}Te, ^{95m}Tc, ²⁴⁵Am, ²⁵³Eu, ¹⁸³Re, ¹⁸⁵W, ¹⁵⁹Dy, ^{127m}Te, ¹⁶⁹Yb, ¹⁰⁵Ag, ^{119m}Sn, ¹⁷¹Tm, ¹⁴⁵Pm, ¹⁵³Gd, ¹³³Ba, ¹⁷⁴Ln, ¹⁶³Tm, ¹⁴⁷Eu, ¹⁷⁵Hf and ^{97m}Tc. As an example, a radiation source of 1 mCi activity needs 2.8×10¹⁴ ¹²⁵I atoms. A typical radiation dose is in the range of 60-70 Gy. Methods of preparing radiation sources have been described in WO 03/054923, incorporated by reference.

External radiation sources may also be used in the brachytherapy described herein, such as, but not limited to, synchrotron radiation sources, UV or laser radiation.

The heavy metal-containing nano-liposome particles of the invention are therefore for use in a method of brachytherapy treatment of immune-related disorders and particularly of malignant and non-malignant proliferative disorders. For example, solid tumors such as carcinoma, sarcoma and melanoma may be treated with the heavy metal-containing nano-liposome particles of the invention, particularly gold- or platinum-containing particles, alone or in combination with brachytherapy, per se or comprised in a pharmaceutical composition or medicament.

It is to be therefore understood that the gold- or platinum-containing nano-liposome particles of the invention, or compositions comprising thereof by themselves or in combination with radioactive source for brachytherapy, are useful for treating or inhibiting tumors at all stages, namely tumor formation, primary tumors, tumor progression or tumor metastasis.

As used herein to describe the present invention, “malignant proliferative disorder”, “cancer”, “tumor” and “malignancy” all relate equivalently to a hyperplasia of a tissue or organ. In general, the heavy metal-containing nano-liposome particles of the invention, or compositions comprising thereof, are to be used in the treatment of solid tumors, for example, carcinoma, melanoma, sarcoma, and lymphoma.

The present invention thereby provides a method of treatment of cancer, proliferative malignancy, or inflammation, said method comprising administering a therapeutically effective amount of heavy metal-, such as gold, iron or platinum, containing nano-liposome particles of the invention, or compositions comprising thereof, to a subject in need.

The term “effective amount” means an amount necessary to achieve a selected result, which at present, involves the amount of heavy metal-containing nano-particles necessary for treating cancer or proliferative malignant or non-malignant disorders, or more specifically, for killing cancerous cells or for treating inflammation.

Said therapeutic effective amount, or dosing, is dependent on severity and responsiveness of the disease state to be treated, with the course of treatment lasting one hour to several hours, or until a cure is effected or a diminution of the disease state is achieved. Persons of ordinary skill can readily determine optimum dosages, dosing methodologies and repetition rates. Optimum dosages may vary depending on the relative potency of individual heavy metal-containing nano-liposome particles of the invention, or compositions comprising thereof, and can generally be estimated based on EC₅₀, found to be effective in in vitro as well as in in vivo animal models. Persons of ordinary skill in the art can easily estimate repetition rates for dosing based on measured residence times, concentrations, and adjustment to the employed seed and dose of irradiation in case of brachytherapy, or other considerations when using for inflammatory diseases.

The terms “treat, treating or treatment” as used herein mean ameliorating one or more clinical indicia of disease activity in a patient having cancer or a proliferative malignant or non-malignant disease. “Treatment” refers to therapeutic treatment.

By “patient” or “subject in need” is meant any mammal for which cancer or anti-inflammatory treatment is desired in order to overcome said malignant or non-malignant disease, particularly a human subject.

Usually, a “therapeutically effective amount” is also determined by the severity of the disease in conjunction with the preventive or therapeutic objectives, the route of administration and the patient's general condition (age, sex, weight and other considerations known to the attending physician).

Various methods of administration may be used for delivering the heavy metal-nano-particles of the invention to a subject in need. Heavy metal-nano-particles may be delivered via intravenous (i.v.), intramuscular (i.m.), intragastric (i.g.), intraperitoneal (i.p.), or topical injections, or orally. In order to be effective therapeutically, heavy metal-containing nano-particles should be prepared in a way that would enable their stability in the system following administration. For use in brachytherapy, the liposomes have to be administered so that they are in close proximity to the tumor to be treated.

In a further aspect, the invention relates to the use of the particles of the invention as a composition for the treatment of immune-related disorders. As found by the present inventors and disclosed by Examples 10 and 11, the heavy metal-nano-particles of the invention may be used with no additional irradiation or radioactive source for the treatment of immune-related disorders such as inflammatory disorders, for example, arthritis. Surprisingly, these particles were found effective also for the treatment of malignant disorders such as cancer.

As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, congenital malformations, disabilities, syndromes, symptoms, deviant behaviors, and atypical variations of structure and function, chronic or permanent health defects resulting from disease.

The terms “disease”, “disorder”, “condition” and “illness” are equally used herein.

Therefore, according to a preferred embodiment, the heavy metal-containing nano-particle, particularly containing gold or platinum, or a composition comprising the same, may be used for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extrahepatic bile ducts, ampulla of Vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. It should be noted that also non-solid tumors such as leukemia's and lymphomas may be treated by the particles of the invention.

An “in vivo” treatment, as used herein, refers to a process that takes place within a living organism. An “ex vivo” treatment relates to a process taking place outside of a living organism or body, e.g. the treatment of cells, wherein said treated cells may be returned to the same or to a different living organism.

As mentioned above, a further embodiment of this aspect of the present invention is the use of the heavy metal-containing nano-liposome particles of the invention in the treatment of inflammation in general, and arthritis in particular, especially rheumatoid arthritis, as well as osteoarthritis, bursitis, reactive arthritis, ankylosing spondylitis, psoriatic arthritis, and other arthropathies. One of the major intents of rheumatoid arthritis treatment is to prevent or diminish synovial tissue hyperplasia, because it forms the pannus tissue that irreversibly destroys the cartilage and bone in the affected joint. Effective drugs for treating rheumatoid arthritis have not been developed until the present time and the developed drugs can exhibit limited efficacies. Once arthritis occurs, it causes economic loss as well as severe pain to the patient. Medical treatments of rheumatoid arthritis being used presently are usually based on non-steroidal anti-inflammatory drugs (NSAIDs). These NSAIDs limitedly improve a patient's condition, but cannot prevent the cartilage destruction of joint area or the progress of disease. Moreover, this treatment must be stopped within one year because of serious side effects.

An even further aspect of the present invention is the use of the developed liposomes impregnated with heavy metal ion complexes, such as iron ions, for their delivery to the circulation through the digestive system, via intragastric or oral administration, for example.

The heavy metal-containing nano-liposome particles of the invention are also to be used in the treatment of physiological disorders, particularly those disorders related to the lack of a particular heavy metal.

One example of physiological disorders is disorders caused by the lack of iron. Iron is needed for many enzymes to function normally, so a wide range of symptoms may eventually emerge, either as the secondary result of the anemia (the most common manifestation of iron deficiency), or as other primary results of iron deficiency. Symptoms of iron deficiency include: fatigue, pallor, irritability, weakness, and pica.

Hence, the heavy metal-containing nano-liposomes described herein may be considered as a delivery system for heavy metals into the organism. The most important outcomes of using such a system of delivery for heavy metals include, amongst other: (i) masking the toxicity of such metal ion complexes, particularly the toxicity of gold and platinum; (ii) increasing the concentration of metal ion complexes under physiological conditions; and (iii) facilitation of the delivery and bioavailability of such heavy metal complexes. Outcomes (ii) and (iii) are especially advantageous since noble elements, particularly gold and platinum, have the tendency to be reduced and appear in their atomic state, thus normally they are less available physiologically.

As used in the specification and the appended claims and in accordance with long-standing patent Law practice, the singular forms “a” “an” and “the” generally mean “at least one”, “one or more”, and other plural references unless the context clearly dictates otherwise. Thus, for example “a cell”, “a peptide” and “an immune modulator agent” include mixture of cells, one or more peptides and a plurality of adjuvants of the type described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLE

### Experimental Procedures

### General Methods of Molecular Biology

A number of methods of the molecular biology art are not detailed herein, as they are well known to the person of skill in the art. Such methods include PCR, expression of cDNAs, transfection of mammalian cells, and the like. Textbooks describing such methods are, e.g., Sambrook et al. (1989) Molecular Cloning, A Laboratory Manual, Cold Spring Harbor Laboratory, ISBN: 0879693096; F. M. Ausubel (1988) Current Protocols in Molecular Biology, ISBN: 047150338X, John Wiley & Sons, Inc. Furthermore, a number of immunological techniques are not in each instance described herein in detail, like for example Western Blot, as they are well known to the person of skill in the art. See, e.g., Harlow and Lane (1988) Antibodies: a laboratory manual, Cold Spring Harbor Laboratory.

### Cell Culture

The cells used in this study were: the CNS-1 type, obtained from rat astrocytes; and the KHJJ line, from breast cancer.

### Example 1

### Preparation of a Mixture of Peg-PL and Peg-DPL in a Water System

Phosphatidyl choline (20 g, PC 98%, Lipoid, Germany) was dissolved in 200 ml of diethyl ether. A water solution (200 ml) containing 50g of PEG of various molecular weights, PEG-200, PEG-400, PEG-600, PEG-1000, PEG-1500, PEG-2000, PEG-4000, PEG-8000 or PEG-12000 (all purchased from Sigma Aldrich®, Israel), 0.5 g of calcium chloride and 10 g of NaCl was prepared. The pH of the solution was preferably adjusted to a value of between pH5 and pH8. Both solutions were charged into a mechanically or magnetically stirred reactor. The trans-phosphatidylation reaction was initiated by the addition of PLD (0.5 g, Meito Sangyo, Japan). The reaction mixture was vigorously stirred for 48 h at 30° C. After completion of the reaction, the mixture was separated into two phases, an organic and an aqueous phase. The organic phase was separated off, using a separation funnel and then washed twice with 200 ml of a solution of water containing EDTA (2 g), at pH 8. The organic phase collected was washed twice again, with 200 ml of distilled water. The organic phase was then dried with anhydrous sodium sulfate, and the ether removed by evaporation under vacuum, yielding 18 g of a mixture of PEG-PL, PEG-DPL and non-reacted phosphatidylcholine.

### Example 2

### Preparation of a Mixture of Peg-PL and Peg-DPL in Organic System

This reaction was performed in two steps:
a. PLD and substrate immobilization: PLD (1 g, Meito Sangyo, Japan) was solubilized in acetate buffer (50 ml) of pH 6.5. The enzyme solution was shaken at room temperature with 10 g enzyme support adsorbent matrix (Amberlite A7, Rhom and Haas, USA) for 5 hours. Although Amberlite A7 was used in this example, silica gel, Celite, ion exchange resin or any adsorbent matrix may be used. The immobilized enzyme was then either filtered off or the solution was decanted to obtain enzyme-loaded wet particles. The wet immobilized enzyme preparation was introduced into a PEG water solution (15 g PEG-400, Sigma-Aldrich, Israel in 15 ml distilled water). The mixture was shaken at room temperature for 2 hours, and then decanted to obtain the wet support loaded with PLD and PEG.
b. The support loaded with PLD and PEG-400 (10 g) which was obtained in step (a) was added into an organic solution of ether (50 ml) containing phosphatidyl choline (5 g). The mixture was magnetically or mechanically stirred at 30° C. for 48 h. The organic phase was decanted, washed with two portions of distilled water (each 100 ml). The ether was evaporated to yield a mixture of phospholipids containing PEG-PL (30-40%) and PEG-DPL (30-40%) and the unreacted PC (5-30%). This product was used as is for the formation of liposomes or washed with EDTA solution to remove the calcium ions.

### Example 3

### Preparation of Stabilized Modified-Gold Complexes

Gold trichloride (1g of HAuCl₄.3H₂O, Sigma Aldrich, USA) was dissolved in 50 ml distilled water to give a yellowish solution of acidic pH (below 4). N-Acetyl cysteine (0.5 g of Sigma) was added into the gold solution to yield a precipitate due to formation of water-insoluble gold N-acetyl cysteine complex, at pH values below 6. The pH of this mixture was adjusted to a value above 4.5 with 0.1 M sodium hydroxide, yielding a transparent solution of N-acetyl cysteine-modified gold complex of high solubility in water at pH values above 4.5. This procedure was repeated with different molar ratios of N-acetyl cysteine/gold trichloride, typically at molar ratios of 0.5:1, 1:1, 2:1, 3:1, 4:1, and 6:1, respectively. In all experiments transparent solutions of water soluble complexes were obtained, when the pH of the solution was adjusted to pH values above 4.5. Similarly, gold complexes were also modified with cysteine, methionine, glutathione, amino thiols, thio-carboxylic acids, diamines, amino acids and other organic ligands capable of binding gold to produce water-soluble gold complexes.

### Example 4

### Preparation of Nano-Liposomes Loaded with Water-Soluble Gold Complexes

Cholesterol (200 mg, Sigma-Aldrich, Israel) and a mixture of the phospholipids product (0.8 g) obtained from Example 1 or Example 2 (typically, a composition of 20% PC, 50% PEG-PL and 30% PEG-DPL) were dissolved in diethyl ether or any organic solvent, e.g. chloroform, dichloromethane, ethyl acetate, di-iso-propyl ether, iso-propanol or a solution of their mixtures at different ratios, contained in a round bottom flask. The organic solvent was evaporated under vacuum at 50° C. to yield a lipid film. The lipid film contained in the flask was dried in a desiccator overnight to remove residual organic solvent and water. A solution of N-acetyl cysteine-modified gold complex (10 ml), as obtained in Example 3, was added into the lipid film. The mixture was spun in a rotary evaporator for 1 hour at 50° C., to allow efficient hydration of the lipid film. The mixture was stirred vigorously, and then homogenized with a high-speed homogenizer to obtain large multi-lamellar vesicles. Ten cycles of freeze-thaw were applied on the liposome solution in order to break the formed multilamellar vesicles (MLV) into smaller vesicles. In order to obtain nano-liposomes, the mixture was sized by multiple extrusions through three stacked membranes of pore sizes 100, 50 and 30 nm, using a medium pressure extruder (Avanti Polar Lipids, USA). TEM (Transmission Electron Microscopy) analysis revealed that the average size of the formed liposomes ranged from 30 to 50 nm. Non-confined water soluble complexes were removed from the liposomes solution by applying Ultra-filtration membrane system or membrane dialysis overnight. The obtained liposome solution was transparent and stable for several weeks.

### Example 5

### Preparation of Gold-Containing Nano-Liposomes by Sonication or Microfluidization

Another mode to prepare the nano-liposomes containing water soluble gold complexes described in this invention may be carried out using other techniques such as sonication or microfluidization. Typically, the MLV solution obtained in Example 4 was further treated with a microfluidizer at a pressure typically in the range of 100-2600 bar for 10 cycles at room temperature, or using a bath and probe tip sonicator at room temperature. Size distribution analysis for the liposomes produced using both techniques showed that the average size of the nano-liposomes was in the range of from 15 nm to 60 nm.

### Example 6

### Preparation of Nano-Liposomes Loaded with Nano-Size Gold Particles

Cholesterol (200 mg, Sigma-Aldrich, Israel) and a mixture (0.8 g) of phospholipids product obtained from Example 1 or Example 2 (typically, a composition of 20% PC, 50% PEG-PL and 30% PEG-DPL) were dissolved in ether, iso-propanol or any organic solvent, e.g. chloroform, dichloromethane, ethyl acetate, di-iso-propyl ether or a solution of their mixtures at different ratios, contained in a round bottom flask. The organic solvent was evaporated under vacuum at 50° C. to yield a lipid film. The lipid film contained in the flask was dried in a desiccator overnight to remove residual organic solvent and water. A solution of N-acetyl cysteine-modified gold complex (10 ml) obtained in Example 3 was added into the lipid film. The mixture was spun in rotary evaporator for 1 hour at 50° C., to allow efficient hydration of the lipid film. The mixture was then stirred vigorously, and homogenized with a high-speed homogenizer to obtain large multi-lamellar vesicles. Ten freeze-thaw cycles were applied on the liposome solution in order to break multilamellar vesicles into smaller vesicles. In order to obtain nano-liposomes the mixture was sized by multiple extrusion through three stacked membranes of pore sizes 100, 50 and 30 nm, using a medium pressure extruder (Avanti Polar Lipids, USA). TEM analysis revealed that the average size of the formed liposomes ranged from 30 to 50 nm. The obtained liposome solution was transparent and stable for several weeks. The liposome solution was mixed with a solution of sodium carbonate (0.1 M) and trisodium citrate (0.2 M). The solution was incubated for 5 hours at 50° C. During this time the formation of nano-gold particles was visually observed by the change of color of the solution or by following the absorbance at different visible wave lengths. Liposomes containing nano-particles of gold in their inner core are obtained by applying gel chromatography, such as using a Sephacryl SF 1000 column (Pharmacia) and HEPES/NaCl buffer as the eluent, in order to remove non-confined gold particles. Gold complexes are also reduced using other reducing agents, such as borohydride, hydrazine and others, in order to yield gold nano-particles.

### Example 7

Delivery of Gold-Containing Liposomes to CNS-1 Type Cells (In-Vitro)

Four types of gold-containing liposomes and a 2 mg/ml gold-containing solution (without liposomes, used as control) were added (200 μl/plate) to CNS-1 type cells for 24 hours. The cells were then washed, counted and prepared according to the standard protocol for solubilization in aqua regia solution for analysis of metals in order to measure the amount of gold that entered the cells.

The four types of liposomes were as follows:

Type 1—10% PL, 1 mg/ml water soluble gold complexes, following dialysis;
Type 2—10% PL, 1 mg/ml water soluble gold complexes, no dialysis;
Type 3—10% PL, 2 mg/ml water soluble gold complexes, following dialysis;
Type 4—10% PL, 2 mg/ml water soluble gold complexes, no dialysis.

The results are shown in Table 1 below.

TABLE 1
Number of gold atoms per cell using CNS-1 type cells in vitro.

| Tube | | Gold atoms |
|---|---|---|
| No. | Sample | per cell |
| 1 | Control | 0 |
| 2 | Control | 0 |
| 3 | Control | 0 |
| 4 | Type 1 Liposomes | 3.53E+10 |
| 5 | Type 1 Liposomes | 2.03E+10 |
| 6 | Type 1 Liposomes | 1.90E+10 |
| 7 | Type 2 Liposomes | 1.57E+10 |
| 8 | Type 2 Liposomes | 1.28E+10 |
| 9 | Type 2 Liposomes | 1.99E+10 |
| 10 | Type 3 Liposomes | 2.41E+09 |
| 11 | Type 3 Liposomes | 1.43E+09 |
| 12 | Type 4 Liposomes | 1.88E+09 |
| 13 | Type 4 Liposomes | 2.46E+09 |
| 14 | Type 4 Liposomes | 2.04E+09 |
| 15 | Au 2 mg/ml | 2.41E+10 |
| 16 | Au 2 mg/ml | 2.12E+10 |
| 17 | Au 2 mg/ml | 2.73E+10 |

The results of the experiments presented in Table 1 show that water soluble gold complexes incorporated in liposomes can diffuse from the medium and accumulate in the cells to reach concentrations of higher than 1×10⁸ atoms of gold per each cell. Also, the results showed that more than 90% of the cell populations in the control experiments and also in experiments where cells were subjected to gold-containing liposomes were alive. In the other hand, more than 80% of the cell populations which were subjected to solutions containing free gold complexes died. This result suggests that gold complexes are toxic when provided free to the cells, while the toxicity of gold is masked by confinement into liposomes.

### Example 8

### Delivery of Gold to Tumor-Bearing Mice using Nano-Liposomes Loaded with Gold Complexes

Table 2 shows gold concentrations achieved in tumor cells after injection of gold-containing liposomes to mice having been injected with tumor cells of the KHJJ type (breast cancer) in their legs. The liposomes were injected via intraperitoneal (i.p., n=2), intravenously (i.v., n=3), or directly into the tumor (n=5). The volume of liposome solution injected via i.p. was 200 μl, while via i.v., it was 1000. Samples from control experiments were treated similarly in order to determine native gold concentration in cells without injection of the liposome solution. Gold concentration was determined by taking samples from the tumor cells (about 100 mg) which were dissolved in aqua regia solution (HNO₃ and HCl) and diluted appropriately with double-distilled (dd) water.

The concentration of gold in each sample was determined by ICP (Inductivity Coupled Plasma Spectroscopy) where the threshold value is 30 ppb. This means that in samples containing less than 30 ppb, the ICP reads 30 ppb of gold.

The concentration of gold in the liposome solution was 3.25 mg/ml. It can be seen that concentrations of higher than 1×10⁸ atoms of gold per cancerous cell were reached when the liposome solution was injected topically into the tumor region, usually in the surrounding tissue.

TABLE 2
Gold concentrations which have been administered to cancer
cells in mice using nano-liposomes loaded with water soluble
acetyl cysteine-modified gold (gold-containing nano-liposomes)
through i.p., i.v. and topical injection.

| Sample | Gold atoms per cell |
|---|---|
| Control | 0 |
| Control | 0 |
| Tumor-200 μl ip-24 h | 3.31E+07 |
| Tumor-200 μl ip-24 h | 4.93E+07 |
| Tumor-200 μl iv-24 h | 2.73E+07 |
| Tumor-100 μl iv-24 h | 4.08E+06 |
| Tumor-100 μl iv-24 h | 1.28E+07 |
| Tumor-50 μl injection | 1.27E+08 |
| to tumor-24 h | |
| Tumor-50 μl injection | 1.22E+08 |
| to tumor-24 h | |
| Tumor-50 μl injection | 1.40E+08 |
| to tumor-24 h | |
| Tumor-50 μl injection | 9.01E+07 |
| to tumor-24 h | |
| Tumor-10 μl injection | 5.12E+08 |
| to tumor-24 h | |

The gold concentration in the liposomes solution was 3.25 mg/ml.

### Delivery of Gold-Containing Liposomes to Animals

Balb/C mice which had tumors on their thighs (KHJJ line, breast cancer) were used in these experiments. The mice were injected i.p. with 200 μl of liposomes containing 2 mg of gold atoms per/ml solution, and after 24 hours the animals were dissected and various internal organs were harvested, which included the tumor, brain, heart, lungs, spleen, liver, kidneys, and blood. The organs were rinsed and prepared for ICP-MS analysis. In control experiments, mice were injected with a solution containing 2 mg of gold/ml without confinement in liposomes.

Tables 3 and 4 below show the results of the organ analysis of the mice treated i.p. with 200 μl of gold solution as follows:

1. A solution of gold chloride of 2 mg/ml (2000). The gold was not confined in liposomes.
2. A gold-containing liposome solution (2000) of gold content of 2 mg/ml.

TABLE 3
Number of gold atoms per cell in different organs
of mice treated with a gold chloride solution containing
2 mg of gold/ml solution (control experiment).

| Organ | Gold atoms per cell |
|---|---|
| Blood | 1.37E+08 |
| Kidneys | 1.58E+09 |
| Liver | 1.88E+08 |
| Spleen | 3.34E+08 |
| Heart | 1.63E+09 |
| Lungs | 1.36E+08 |
| Brain | 1.62E+07 |
| Tumor | 1.10E+08 |
| Bladder | 1.30E+10 |

The results of the experiments presented in Table 3 show that non-confined gold is delivered to all organs and accumulates in the cells at different concentrations depending on the type of the organ. Also, it was observed that mice which were injected with free gold solution died after 24 hours due to the toxicity of gold.

TABLE 4
Number of gold atoms per cell in different organs of mice treated
with a gold-containing liposome solution of 2 mg/ml of gold.

| Organ | Gold atoms per cell |
|---|---|
| Blood | 1.93E+07 |
| Kidneys | 4.81E+08 |
| Liver | 2.13E+08 |
| Spleen | 1.19E+08 |
| Heart | 2.30E+07 |
| Lungs | 5.21E+07 |
| Brain | 6.88E+06 |
| Tumor | 8.02E+08 |

The results of this experiment show that mice injected with similar concentrations of gold which is confined in liposomes, survived. This result indicates that the toxicity of gold can be reduced, or overcome, when this heavy metal is confined into liposomes according to the present invention.

Also, the results presented in Table 4 show clearly that water-soluble gold complexes or gold nano-particles loaded in liposomes can be delivered efficiently to the cancerous cells through i.p. injections. Following the procedure developed in this invention it can also be seen that the delivery of desired concentrations of metal ions or atoms can be achieved through i.p., i.v. or through topical injections in the cancerous tissue surrounding (tissue around the tumor) in order to expedite the desired therapeutic effect.

### Example 9

Brachytherapy Treatment of a Prostate Tumor using the Gold-Containing Nano-Liposomes of the Invention

Before starting the treatment, it is preferable to perform three-dimensional imaging of the tumor of the patient to be treated, in order to get a picture of the morphology of the tumor and its position with regards to the surrounding normal tissue. This should assist in the calculations of radiation dose, as well as with determining the optimal positioning of the bracytherapy seed. The seeds are implanted interstitially in the prostate tumor. On the next day, the corresponding optimal dose of the gold-containing nano-liposome particles is administered intravenously to the patient. If necessary, the gold-containing nano-liposome particles may be delivered several times during the course of the radiation.

### Example 10

### Use of Gold-Containing Liposomes in the Treatment of Cancer

Balb/C mice (3 groups of 10 mice each) which had tumors in their thighs (KHJJ line, breast cancer) were used in these experiments to show the therapeutic effect of water-soluble gold complexes impregnated in liposomes.

The first experimental group was a control group of mice with an induced tumor without any treatment. The second experimental group consisted of each mouse being injected i.p. with 200 μl of liposomes containing 10 mg of water-soluble gold complex as prepared in Example 4. The third experimental group consisted of each mouse being injected i.p. with 200 μl of liposomes containing 10 mg of water-soluble gold complex as prepared in Example 4, and being treated with brachytherapy treatment. Tin¹⁷⁰ seeds were implanted into tumors 24 hours post injection of the liposomes. Results of the third group are summarized in Table 5 below.

TABLE 5
Mice with solid tumor undergo treatment with
gold-containing liposomes and brachytherapy.

| Treatment | Number of animals | % of cure |
|---|---|---|
| Control | 20 | 0 |
| Dummy seeds | 20 | 0 |
| I¹²⁵ seeds | 16 | 20 |
| Tm¹⁷⁰ seeds | 20 | 60 |
| Tm¹⁷⁰ seeds + Au | 12 | 75 |

The results of the third experimental group show that use of Tm¹⁷⁰ seeds was much more efficient (60% cure) than use of I¹²⁵ seeds (20%), which is usually the radiation source of choice.

In sum, the tumor in the control group (first experimental group) did not change after one week. Surprisingly, the inventors observed that in 80% of the mice of the second experimental group which were injected with liposomes containing water-soluble gold complex the tumors were converted after one week to a lump of pus. Removal of the pus from the mice thighs with the aid of a syringe resulted in curing the mice group from cancer. Tumors in the third group of mice, which received bracytherapy, also disappeared after one week (in 75% of the mice treated) however without the appearance of a lump of pus.

These surprising results show that the water-soluble gold-containing liposomes can also be used by themselves, without irradiation, for the efficient and safe treatment of immune related disorders such as cancer and inflammatory conditions.

The results of the present invention further exemplify the feasibility of using the gold-containing liposomes in combination with an appropriate source of radiation implanted in the diseased area for brachytherapy of cancer.

### Example 11

Treatment of Rheumatoid Arthritis using the Gold-Containing Nano-Liposomes of the Invention

The corresponding optimal dose of the gold-containing nano-liposome particles is administered intravenously to the patient, or directly into the tissue mostly affected by the rheumatoid condition. If necessary, the gold-containing nano-liposome particles may be delivered several times during the course of the treatment.

### Example 12

### Preparation of Nano-Liposomes Loaded with Water-Soluble Platinum Complexes

The same procedure described in Example 3 was adopted for the preparation of dichloro-dicysteine platinate (Pt⁺²) complex, starting from potassium tetrachloroplatinate. Liposomes containing dichloro-dicysteine platinate were prepared according to Example 4. Such dichloro-dicysteine platinate-containing liposomes are used for reducing the toxicity of platinum complexes, improving their solubility under physiological conditions and thus improving their delivery to cancerous cells for cancer therapy.

### Example 13

### Preparation of Nano-Liposomes Loaded with Water-Soluble Iron Complexes

The same procedure described in Example 3 was adopted for the preparation of divalent iron ions (Fe⁺²) modified with cysteine at a molar ratio of 1:4. The ionic iron metal species used were mainly FeCl₂, FeSO₄ and FeCl₃. Liposomes containing iron ions were prepared according to Example 4. Such iron-containing liposomes are used for the efficient delivery of iron ions to the circulation through the digestive system, usually through intra-gastric or oral administration.

While this invention has been described in terms of some specific examples, many modifications and variations are possible. It is therefore understood that within the scope of the appended claims, the invention may be realized otherwise than as specifically described.

1-61. (canceled)

62. A method of preparing a stable, water-soluble heavy metal complex in the presence of polar lipids or a reducing factor, preferably citrate, amine, thiol, reducing agent present at physiological conditions or derivatives thereof, said method comprising the steps of: (a) dissolving an ionic heavy metal species in water to obtain a transparent ionic metal solution; (b) adding a ligand capable of binding heavy metal ions, preferably N-acyl cysteine or cysteine, to produce a heavy metal complex which is water insoluble or water soluble at pH values below 4.5; and (c) adjusting the pH of the mixture of step (b) to a value of at least 4.5 with a suitable base, preferably a sodium hydroxide solution, yielding a heavy metal complex of high solubility in water solutions of pH 5 or higher.

63. The method of claim 62, wherein said heavy metal is gold, platinum, iron, silver, copper, nickel, palladium, iridium, titanium or another heavy metal of therapeutic use.

64. A method of preparing a stable, water-soluble gold complex in the presence of polar lipids or a reducing factor, said method comprising the steps of: (a) dissolving an ionic gold species selected from HAuCl₄ and MAuCl₄, wherein M is an alkali metal cation, preferably gold trichloride, in water to obtain a transparent ionic gold solution; (b) adding a ligand capable of binding gold ions, preferably N-acyl cysteine, to produce a gold complex which is water insoluble at pH values below 4.5; and (c) adjusting the pH of the mixture of step (b) to a value of at least 4.5 with a suitable base, preferably a sodium hydroxide solution, yielding a gold complex of high solubility in water solutions of pH 5 or higher.

65. The method according to claim 64, wherein said reducing factor is a polar lipid or a mild reducing factor, which may be citrate, amine, thiol or a reducing agent present at physiological conditions.

66. A water-soluble heavy metal complex comprising an ionic heavy metal species and one of N-acyl cysteine or other ligand, said complex is water soluble and resistant to chemical reduction by mild chemical reducing agents in aqueous solutions having a pH higher than 4, and particularly at physiological pH values, wherein the molar ratio of said heavy metal ionic species to said N-acyl cysteine or other ligand is at least 0.5:1.

67. A water-soluble gold complex comprising an ionic gold species and N-acyl cysteine, said complex is water soluble and resistant to chemical reduction by mild chemical reducing agents in aqueous solutions having a pH higher than 4, and particularly at physiological pH values, wherein the molar ratio of said gold ionic species to said N-acyl cysteine is at least 0.5:1.

68. A method of preparing nano-liposomes comprising ionic or atomic heavy metal, said method comprising the steps of: (a) mixing the heavy metal complex of claim 66 or 67 with a liposome-forming surface-active material; (b) homogenizing the mixture of step (a) to obtain large multilamellar vesicles; (c) optionally, applying freeze-thaw cycles to the large multilamellar vesicles to generate smaller vesicles; and (d) sizing the mixture of step (b) or (c) by multiple extrusions, sonication or using a microfluidizer to yield heavy metal-containing nano-liposomes from 15 to 150 nm in size.

69. The method of claim 68, wherein said liposome-forming surface-active material is a phospholipid, ceramide, sphingomyelin or cholesterol, and wherein said phospholipid is phosphatidyl choline, phosphatidyl ethanolamine, phosphatidyl glycerol, phosphatidyl inositol, phosphatidyl serine, a PEG-phospholipid, an ether phospholipid or mixture thereof.

70. The method of claim 68, wherein said heavy metal is gold, platinum, iron, silver, copper, nickel, palladium, iridium, titanium or another heavy metal of therapeutic use.

71. A method of preparing nano-liposomes comprising ionic heavy metal complexes or heavy metal atoms, said method comprising the steps of: (a) dissolving cholesterol and a mixture of phospholipids, preferably PEG-PL and PEG-DPL, in an organic solvent; (b) evaporating the organic solvent under suitable conditions, preferably under vacuum at 50° C., to yield a lipid film; (c) drying said lipid film obtained in step (b) under suitable conditions to remove residual organic solvent and/or water; (d) mixing the solution of heavy metal complex of claim 66 with the lipid film of step (c) and allowing hydration of said lipid film; (e) homogenizing the mixture of step (d) to obtain large multilamellar vesicles; (f) optionally, applying freeze-thaw cycles to the large multi-lamellar vesicles to generate smaller vesicles; and (g) sizing the mixture of step (e) or (f) by multiple extrusions, sonication or using a microfluidizer to yield heavy metal-containing nano-liposomes from 15 to 150 nm in size.

72. A heavy metal-containing nano-liposome particle comprising a complex of heavy metal ions or heavy metal atoms and an organic ligand, entrapped in an organic layer comprising pegylated phospholipids and di-phospholipids or any liposome-forming surface-active ingredient, wherein said heavy metal complex is stable at physiological pH, in vivo or in vitro.

73. The heavy metal-containing nano-liposome particle of claim 72, wherein said heavy metal is gold, platinum or iron.

74. A composition comprising the heavy metal nano-liposome particle of claim 72 and a pharmaceutically acceptable additive, carrier, buffer, stabilizer, excipient or mixture thereof, and optionally further comprising at least one therapeutic agent.

75. A kit for the treatment of a patient suffering from an immune-related disorder, wherein said kit comprises: (a) the heavy metal-containing nano-liposome particles of claim 72, or a composition comprising said particles; (b) means for administering said particles to the patient; and (c) instructions for use.

76. The kit of claim 75, wherein said administration means comprises a means for intravenous, intraperitoneal, intragastric, oral, intra-tumoral, topical or combination thereof administration.

77. A kit for use in brachytherapy of an immune-related disorder, wherein said kit comprises: (a) the heavy metal-containing nano-liposome particles of claim 72 or the composition of claim 74; (b) implantable seeds of a radioactive source or an external radioactive source, wherein said source is selected from the group consisting of Thulium (¹⁷⁰Tm), ¹⁰³Pd, ¹⁴⁵Sm, ¹²⁵I, a mixture of ¹²⁵I and ¹²⁷I, ²³⁴Th, ^{93m}Nb, ¹⁴⁰Ba, ¹⁹⁵Au, ¹⁴⁴Ce, ¹²⁵̂Te, ^{93m}Tc, ²⁴⁵Am, ²⁵³Eu, ¹⁸³Re, ¹⁸⁵W, ¹⁵⁹Dy, ^{127m}Te, ¹⁶⁹Yb, ¹⁰⁵Ag, ^{119m}Sn, ¹⁷¹Tm, ¹⁵³Gd, ¹³³Ba, ¹⁷⁴Ln, ¹⁶³Tm, ¹⁴⁷Eu, ¹⁷⁵Hf and ^{97m}Tc, wherein said radioactive source promotes an Auger effect in combination with gold; (c) means for delivering said heavy metal-containing nano-liposome particles to a patient in need of brachytherapy; (d) means for delivering said implantable seeds to said patient; and (e) instructions for use.

78. The brachytherapy kit of claim 77, wherein said immune-related disorder is a malignant or a non-malignant proliferative disorder.

79. A method of brachytherapy treatment of a malignant proliferative disorder comprising the step of administering to a subject in need thereof a therapeutically effective amount of: (a) the heavy metal-containing nano-liposome particles of claim 72 or the composition of claim 74; and (b) implantable seeds of a radioactive source, wherein said source is selected from the group consisting of Thulium (¹⁷⁰Tm), ¹⁰³Pd, ¹⁴⁵Sm, ¹²⁵I, a mixture of ¹²⁵I and ²³⁴Th, ^{93m}Nb, ¹⁴⁰Ba, ¹⁹⁵Au, ¹⁴⁴Ce, ^{125m}Te, ^{93m}Tc, ²⁴⁵Am, ²⁵³Eu, ¹⁸³Re, ¹⁸⁵W, ¹⁵⁹Dy, ¹²⁷̂Te, i69gammab, 105Ag, ¹¹̂Sn, “¹Tm, ¹⁴⁵ Pm, ¹⁵³Gd, ¹³³Ba, ¹⁷⁴Ln, ¹⁶³Tm, ¹⁷⁵Hf and ^{97m}Tc, wherein said radioactive source promotes an Auger effect in combination with gold.

80. The method of claim 79, wherein said administration step comprises a means for intravenous, intraperitoneal, intragastric, oral, intra-tumoral, topical or combination thereof administration.
